SEQUENCE LISTING (9 sequences)

SEQ ID NO: 1 - protein, 15 aa, Homo sapiens
CGGRKRRSSRRSAGG

SEQ ID NO: 2 - protein, 15 aa, Homo sapiens
CNVRERQRTQSLNEA

SEQ ID NO: 3 - protein, 12 aa, Homo sapiens
RKRRSSRRSAGG

SEQ ID NO: 4 - protein, 12 aa, Homo sapiens
RKRRSARRSAGG

SEQ ID NO: 5 - protein, 12 aa, Homo sapiens
RKRRSSRRAAGG

SEQ ID NO: 6 - protein, 12 aa, Homo sapiens
RERQRTQSLNEA

SEQ ID NO: 7 - protein, 12 aa, Homo sapiens
RERQRAQSLNEA

SEQ ID NO: 8 - protein, 12 aa, Homo sapiens
RERQRTQALNEA

SEQ ID NO: 9 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

(19) United States
(12) Patent Application Publication
(10) Pub. No.: US 2012/0213801 A1
(43) Pub. Date: Aug. 23, 2012

(54) Phosphorylated Twist1 and cancer

(76) Inventors: Ekaterina Gresko, Zurich (CH); Brian Arthur Hemmings, Bettingen (CH); Anton Vychalkovskiy, Zurich (CH); Gongda Xue, Allschwil (CH)

(21) Appl. No.: 13/504,981

(22) PCT Filed: Oct. 10, 2010
(86) PCT No.: PCT/EP10/66361

§ 371 Date: Apr. 30, 2012

(30) Foreign Application Priority Data
Oct. 30, 2009 (EP) 09174681.8

(51) Int. Cl. A61K 39/395; A61P 35/02; G01N 33/53; A61K 38/02; C07K 2/00

(52) U.S. Cl. 424/172.1; 514/19.3; 530/300; 435/7.1

(57) ABSTRACT

The present application relates to methods for treating cancer in a subject by modulating the phosphorylation of the Serine 42 of Twist1 by administering to said subject a therapeutically effective amount of a modulator of said phosphorylation of the Serine 42 of Twist1. Antibodies, uses methods and biomarkers based on the phosphorylation of the Serine 42 of Twist1 are also provided.

### FIELD OF THE INVENTION

The present invention relates to a method of treating cancer by modulating the phosphorylation of the serine 42 of Twist-1.

### BACKGROUND OF THE INVENTION

Twist-1 is a highly conserved member of a family of regulatory basic helix-loop-helix (bHLH) transcription factors (Thisse, el Messal et al., 1987, Nucleic Acids Res, 15, 3439). bHLH proteins form active dimers with E-box-proteins and bind to a core sequence (CANNTG, referred to as E-box) in the regulatory elements of many lineage-specific genes in muscle, cartilage and osteogenic cells. Germ-line mutations of the Twist-1 gene that result in haploinsufficiency lead to the development of one of the most commonly inherited craniosynostosis conditions, the Saethre-Chotzen syndrome (SCS), which is characterized by premature fusion of cranial sutures and limb abnormalities (Cai and Jabs, 2005, Bioessays, 27, 1102; Ghouzzi, Legeai-Mallet et al., 2001, FEBS Lett, 492, 112; Gripp, Zackai et al., 2000, HumMutat, 15, 150; Yang, Mani et al., 2004, Cell, 117, 927). Expression of Twist-1 has also been implicated in the inhibition of differentiation of various cell lineages including osteoblasts and myoblasts (Bialek, Kern et al., 2004, DevCell, 6, 423; Hayashi, Nimura et al., 2007, JCell Sci, 120, 1350; Spicer, Rhee et al., 1996, Science, 272, 1476).

There are many reports that Twist-1 is involved in oncogenesis in a wide variety of human cancers by inhibiting apoptosis and promoting cell survival after DNA damage or oncogene activation. For example, Twist-1 participates in malignant transformation in neuroblastoma, where it cooperates with the amplified N-Myc oncogene to inhibit p53-mediated apoptosis (Valsesia-Wittmann, Magdeleine et al., 2004, Cancer Cell, 6, 625); reviewed by (Puisieux, Valsesia-Wittmann et al., 2006, BrJCancer, 94, 13). Twist-1 can induce an epithelial mesenchymal-like transition (EMT), proposed to be an important step in tumorogenesis and metastasis (Smit, Geiger et al., 2009, Mol Cell Biol, 29, 3722; Yang et al., 2004, Cell, 117, 927; Yang, Mani et al., 2006, Cancer Res, 66, 4549). A recent study also suggests Twist-1 involvement in tumor progression via direct activation of its transcriptional target YB-1 (Shiota, Izumi et al., 2008, Cancer Res, 68, 98). Twist-1 expression can be regulated by hypoxia-induced HIF-1 via direct binding to the hypoxia-response element (HRE) in the TWIST proximal promoter. This signaling pathway is thought to promote metastasis in response to intratumoral hypoxia (Yang, Wu et al., 2008, NatCell Biol, 10, 295).

Elevated Twist-1 expression is correlated with a poor prognosis and high risk of metastasis in breast, prostate, ovarian, cervical and many others human cancers (Elias, Tozer et al., 2005, Neoplasia, 7, 824; Hosono, Kajiyama et al., 2007, BrJCancer, 96, 314; Kwok, Ling et al., 2005, Cancer Res, 65, 5153; Kyo, Sakaguchi et al., 2006, HumPathol, 37, 431; Mironchik, Winnard et al., 2005, Cancer Res, 65, 10801; Puisieux et al., 2006, BrJCancer, 94, 13; Shibata, Kajiyama et al., 2008, AnnOncol, 19, 81). Recent reports suggest that high levels of Twist-1 confer cancer cells resistance to various chemotherapeutic drugs (Pham, Bubici et al., 2007, MolCell Biol, 27, 3920; Shiota et al., 2008, Cancer Res, 68, 98; Zhang, Wang et al., 2007, IntJCancer, 120, 1891). PKB/Akt protein kinase plays a pivotal role in cell signaling in response to a variety of extracellular stimuli, such as growth factors and cytokines, as well as γ-irradiation (Bozulic, Surucu et al., 2008, Mol Cell, 30, 203). An intact PKB signaling is essential for cell growth and proliferation, whereas loss or gain of the function of this kinase is associated with complex diseases such as type-2 diabetes and cancer (for review see (Fayard, Tintignac et al., 2005, JCell Sci, 118, 5675; Yoeli-Lerner and Toker, 2006, Cell Cycle, 5, 603)). A somatic mutation (El 7K) in the lipid-binding pocket of PKBα was identified recently in human breast, colorectal and ovarian cancers. This mutation resulted in pathological localization of the kinase to the plasma membrane, increasing activation and downstream signaling, that can induce oncogenic transformation of mouse lymphocytes (Carpten, Faber et al., 2007, Nature, 448, 439; Restuccia and Hemmings, 2009, Science, 325, 1083). Many PKB substrates have been identified in the nucleus. PKB phosphorylation of forkhead transcription factors inhibits their transcriptional activity by promoting their association with 14-3-3 regulatory proteins, retention in the cytoplasm and subsequent ubiquitinilation (Biggs, Meisenhelder et al., 1999, ProcNatlAcadSciUSA, 96, 7421; Kops, de Ruiter et al., 1999, Nature, 398, 630). Phosphorylation of the CDK inhibitor p27 impairs its nuclear import and opposes cell cycle arrest (Liang, Zubovitz et al., 2002, NatMed, 8, 1153), while phosphorylation of p21 prevents its nuclear localization and interaction with CDK2 (Zhou, Liao et al., 2001, NatCell Biol, 3, 245). So far, PKB and Twist-1 have not been identified as members of the same signaling cascade.

### SUMMARY OF THE INVENTION

The present inventors noted however that, despite the fact that it had been reported that Twist1 is phosphorylated by PKA (Firulli, Krawchuk et al., 2005, NatGenet, 37, 373), several reports could be indicative of a mutual regulation between Twist1 and PKB. Twist-1 transactivates the PKBβ promoter and a positive association between elevated levels of Twist-1 and PKB β has been found in late-stage breast cancer samples (Cheng, Chan et al., 2007, Cancer Res, 67, 1979). PKB in turn might act as a functional mediator of Twist-1 and is involved in Twist-mediated chemotherapeutic drug resistance (Cheng et al., 2007, Cancer Res, 67, 1979; Zhang et al., 2007, IntJCancer, 120, 1891). Interestingly, SCS resulting from Twist-1 haploinsufficiency displays decreased expression of Cbl ubiquitin ligase, resulting in the accumulation of PI3K and increased PI3K/PKB signaling (Guenou, Kaabeche et al., 2006, AmJPathol, 169, 1303).

https://connect.fmi.ch/,DanaInfo=www.sciencedirect.com+science? ob=ArticleURL& ud=B6 WSN-4W9297H-9& user=5234439& rdoc=1& fmt=& orig=search& sort=d& docanchor=&view=c& acct=C 000006118& version=1& urlVersion=0& userid=5234439&md5=7fa9917c68098913a411c6 d3e9a336dd aff1

The present inventors therefore investigated whether there is an interaction between PKB and Twist1. In the present disclosure, the present inventors show that PKB kinase becomes activated and phosphorylates transcription factor Twist-1 at serine 42 in MCF-7 cells following γ-irradiation and DNA damage induced by adriamycin. The present inventors noted that this posttranslational modification of Twist-1 is necessary for the subsequent decrease in total p53 level and the inhibition of cell cycle arrest and apoptosis via impaired activation of p53 target genes. Moreover, the present inventors found that Twist-1 Ser42 phosphorylation occurs in particular human cancers, especially colorectal, breast, lung and prostate. The results presented in the present disclosure thus provide evidence that Twist-1 is a novel PKB nuclear substrate and establish a link between PKB activation and the downregulation of the p53 tumor suppressor. Moreover, the present inventors also found that Twist1 phosphorylation promotes EMT and metastasis.

The present invention thus encompasses a method for treating cancer in a subject by modulating the phosphorylation of the Serine 42 of Twist1 by administering to said subject a therapeutically effective amount of a modulator of said phosphorylation of the Serine 42 of Twist1. In some embodiments, the phosphorylation of the Serine 42 of Twist1 is modulated by an inhibitor which specifically binds to Twist1 and hinders the phosphorylation of its Serine 42 by PKB., for instance an antibody or a small molecule. In some embodiments of the invention, the subject is a mammal, for instance a human subject. In some embodiments, the method of the invention is performed in vivo, ex vivo or in vitro.

In some embodiments of the invention, the epithelial-mesenchymal transition (EMT) of cancer cells and/or metastasis formation is reduced. In some embodiments, the cancer is a melanoma, a colorectal cancer, a breast cancer, a lung cancer or a prostate cancer.

The present invention also encompasses an antibody or a small molecule specifically binding to Twist1 and hindering the phosphorylation of the Serine 42 of Twist1 by PKB, for use as a medicament to treat cancer, for instance, said antibody specifically binds to an epitope of Twist1, which epitope comprises the Serine 42 of Twist1.

In some embodiments, fragments of the Twist1 protein, which fragments comprise an amino acid corresponding to the Serine 42 of Twist1 and is recognized and phosphorylated by PKB, can be used to modulate the phosphorylation of the Serine 42 of Twist1 by PKB. Example of such fragments are described herein below, for instance those of SEQ ID NO:1, 3 or 5.

The present invention also encompasses a method for the identification of a substance that modulates a PKB signaling pathway, which method comprises the step of assessing the phosphorylation of the Serine 42 of Twist1. Another aspect of the present invention is a method of diagnosing cancer comprising the step of assessing the phosphorylation of the Serine 42 of Twist1. In some embodiments, an increased phosphorylation of the Serine 42 of Twist1 is indicative of a potential epithelial-mesenchymal transition of cancer cells and/or metastasis formation. Moreover, an increased phosphorylation of the Serine 42 of Twist1 can also be indicative of a potential resistance to chemotherapeutic drugs.

A further aspect of the present invention encompasses the use of the phosphorylation of the Serine 42 of Twist1 as a biomarker for cancer or to stratify cancer patients. Moreover, the metastatic potential of the cancer can be assessed according to this aspect of the present invention.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1: Human Twist-1 is phosphorylated in vivo by PKB at Ser42 but not at Ser123.

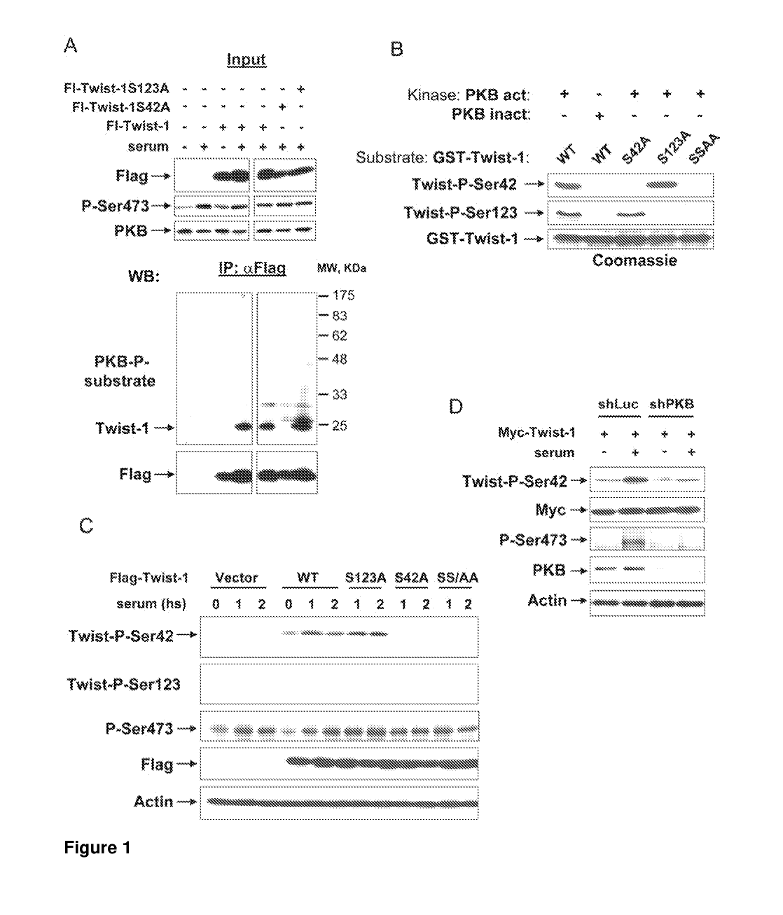

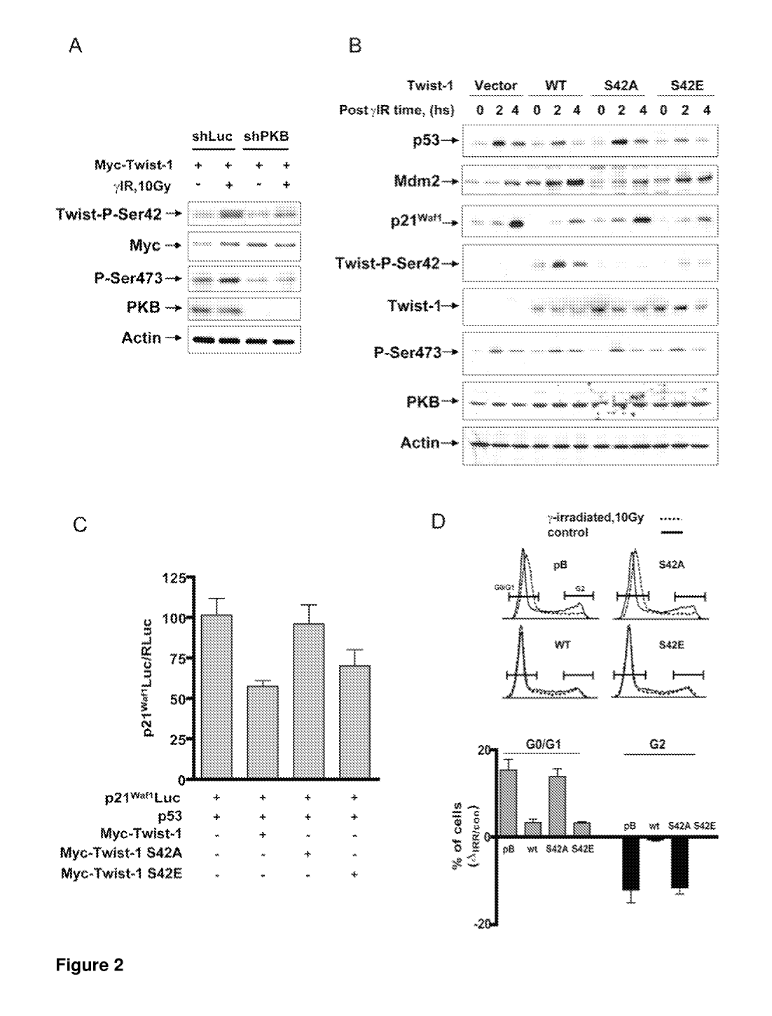

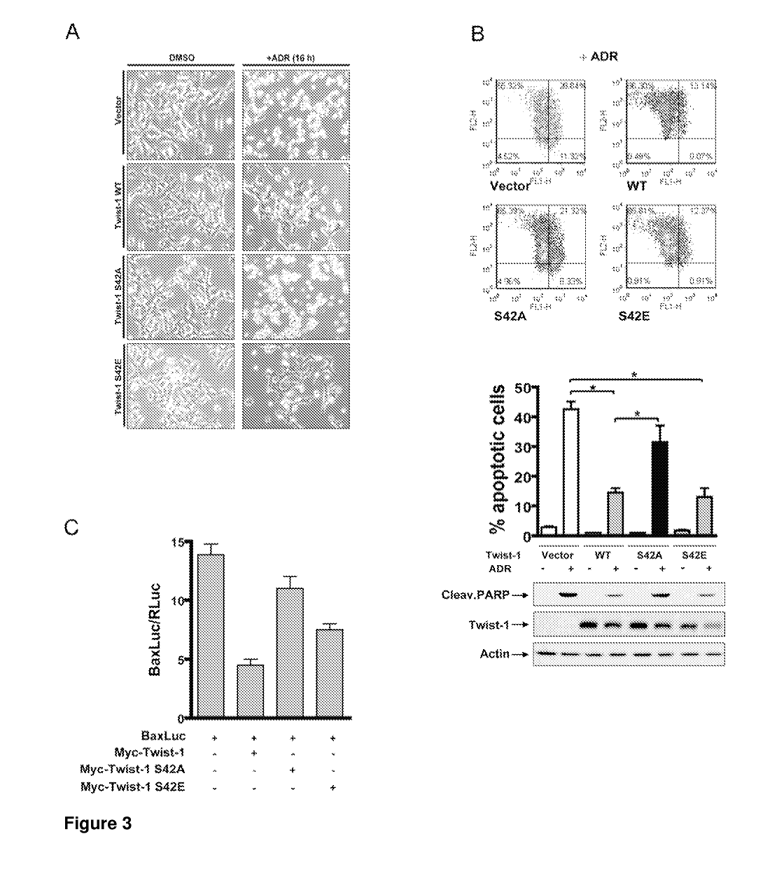

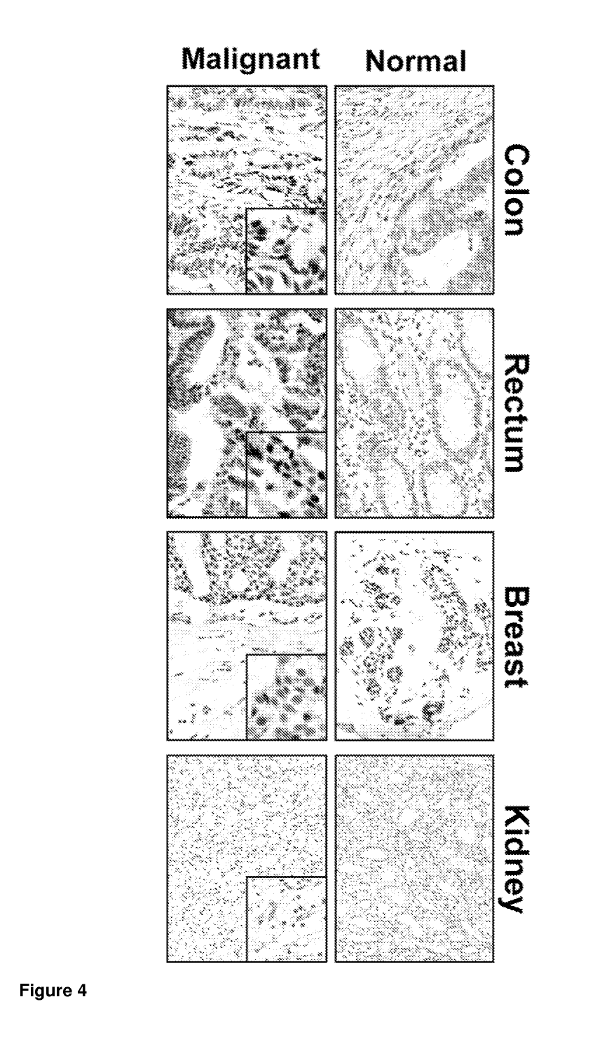

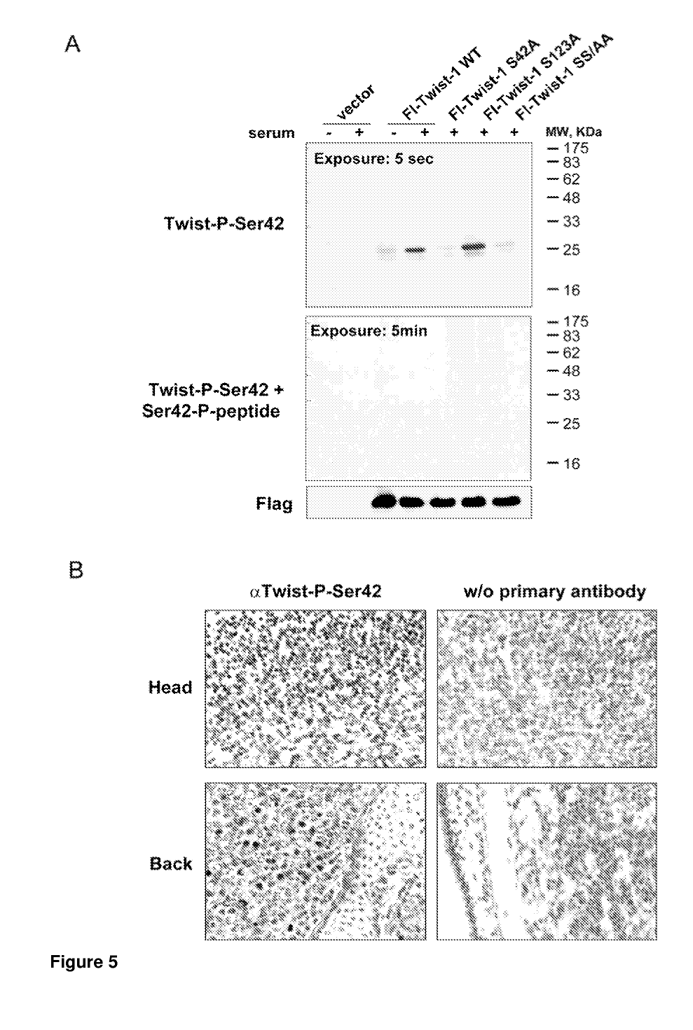

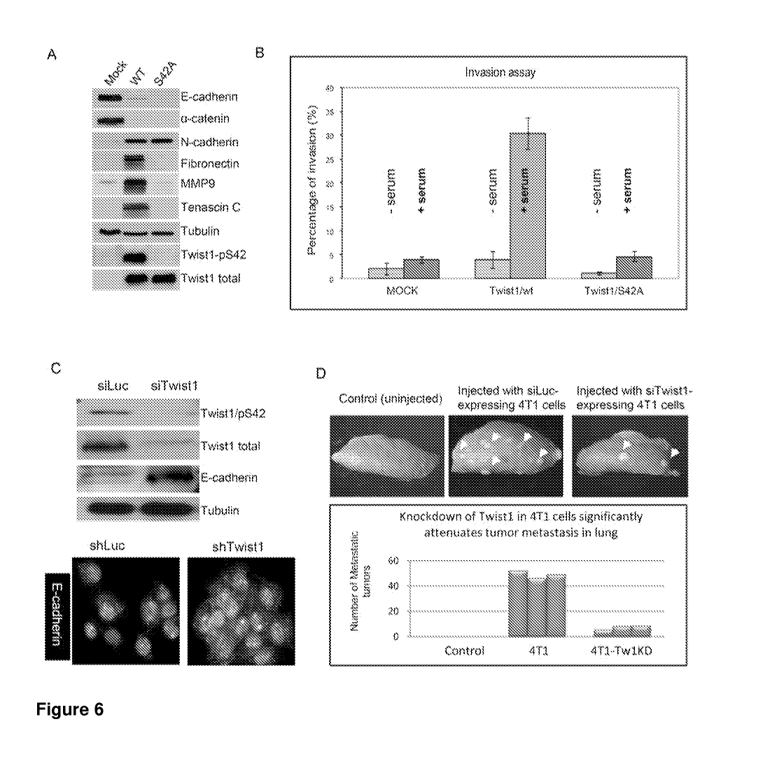

(A) Flag-tagged Twist-1 WT, Ser42A or Ser123A expressed in serum-stimulated HEK293 cells were immunoprecipitated with anti-Flag antibody and the lysates analyzed by Western blotting with the anti-PKB phosphosubstrate antibody. (B) GST-Twist-1 WT and corresponding mutant proteins were phosphorylated in vitro by recombinant PKBβ followed by SDS-PAGE and analysis by Western blotting with the specific anti-Twist-P-Ser42 and anti-Twist-P-Ser123 antibodies. (C) HEK293 cells expressing Flag-tagged Twist-1 WT, Ser42A, Ser123A or the Ser42,123/AA (SS/AA) double mutant were stimulated with serum for the times indicated and analyzed by Western blotting with the anti-Twist-P-Ser42 and anti-Twist-P-Ser123 antibodies. (D) HEK293 cells expressing shRNA against PKB were stimulated with serum; Twist-1 phosphorylation was detected by Western blotting with the anti-Twist-P-Ser42 antibody.

FIG. 2: Phosphorylation of Twist-1 at Ser42 by PKB regulates Twist-1-mediated inhibition of p53 upon DNA damage.

(A) MCF7 cells expressing either control shRNA or shRNA against PKB were γ-irradiated (10 Gy). Cells 2 h post-irradiation were harvested and analyzed by Western blotting. (B) MCF7 cells transfected with empty vector (pB) as a control or stably expressing WT or mutant Twist-1 proteins Ser42Ala (S42A) and Ser42Glu (S42E) were γ-irradiated (10 Gy) and then harvested after the times indicated. The induction of p53, p21 Waf1 and Mdm2 as well as the phosphorylation of Ser42 of Twist-1 and Ser473 of PKB were analyzed by Western blotting. (C) H1299 cells were transfected with a combination of different plasmids as indicated below. At 24 h post-transfection, cells were processed and luciferase activity measured. The results are from duplicate assays from three independent experiments showing means ±standard deviations. (D) MCF7 cells stably expressing Twist-1 or its mutants were 7-irradiated (10 Gy). Cells 24 h post-irradiation were fixed and the cell cycle distribution analyzed by flow cytometry (top). The diagram displays the quantitative differences between the numbers of irradiated and control (non-irradiated) cells in GO/G1 and in G2 (bottom). The results are from three independent experiments expressed as means ±standard deviations.

FIG. 3: Phosphorylation at Ser42 is essential for the anti-apoptotic function of Twist-1.

(A) Images of MCF7 cells transfected with empty vector (pB) or stably expressing WT or mutant Twist-1 proteins after 16 h stimulation with DMSO (control) or with adriamycin (ADR, 10 μM). (B) Cells treated as in (A) were analyzed for depolarization of mitochondrial membrane potential by flow cytometry (top). A quantification summary of three independent experiments is shown at the bottom. Data are means ±standard deviations; asterisk P<0,005. The appearance of cleaved PARP and expression levels of Twist-1 constructs were monitored in parallel by Western blotting. (C) MCF7 cells were transfected with a combination of different plasmids as indicated below. At 24 h post-transfection, cells were treated as in (A) and then assayed for luciferase activity. The results are from duplicate assays in three independent experiments; the data are means ±standard deviations.

FIG. 4: Human cancer in various organs show high levels of Twist-1 Ser42 phosphorylation.

(A) Sections of paraffin embedded tissue microarray slides were analyzed by IHC for the occurrence of Twist-1 phosphorylation with the anti-Twist-P-Ser42 antibody. Images of representative cores from different organs, counterstained with hematoxylin (20× objective).

FIG. 5: Characterization of anti-Twist-P-Ser42 antibody by immunoblotting and immunohistochemistry.

(A) HEK293 cells expressing Flag-tagged Twist-1 WT, Ser42A, Ser123A or Ser42,123/AA (SS/AA) double mutant were stimulated with serum and analyzed by Western blotting with anti-Twist-P-Ser42 antibody alone or with anti-Twist-P-Ser42 antibody pre-incubated with S42 phosphopeptide. (B) Paraffin sections of whole mouse embryos at E14.5 were analyzed for the occurrence of Twist-1 phosphorylation with the anti-Twist-P-Ser42 antibody. Different tissues of embryo are displayed (40× objective), counterstained with hematoxylin.

FIG. 6: Twist1 phosphorylation promotes EMT and metastasis.

A) over expression of wild-type Twist1 and S42A mutant in MDCK cells and both epithelial and mesenchymal markers are analyzed by western blotting (Mock: empty vector); B) Boden-chamber assay: wild-type Twist1 expressing MDCK cells exhibit strongly increased invasiveness phenotype after serum stimulation (Mock: empty vector expressing MDCK cells). C) Knockdown of Twist1 in 4T1 cells restores E-cadherin on plasma membrane. Left panel: endogenous Twist1 is knocked down by shRNA in 4T1 cells and examined by western blot (control: non-specific siRNA targeting luciferase); right panel: after Twist1 knocking down, the level of Ecadherin is dramatically restored at the intercellular junctions. D) Knockdown of Twist1 attenuates tumor metastasis in lung in Balb/c mice. Upper panel: arrows point to the tumor nodules in lung of mice injected with siLuc-expressing 4T1 cells. Lower panel: the plot shows the average number of nodules in lung in 4T-Tw1-knockdown injected mice is 7 compared with 50 in 4T1 injected cells

### DETAILED DESCRIPTION OF THE INVENTION

The present inventors noted that, despite the fact that it had been reported that Twist1 is phosphorylated by PKA (Firulli, Krawchuk et al., 2005, NatGenet, 37, 373), several reports could be indicative of a mutual regulation between Twist1 and PKB. Twist-1 transactivates the PKBβ promoter and a positive association between elevated levels of Twist-1 and PKB β has been found in late-stage breast cancer samples (Cheng, Chan et al., 2007, Cancer Res, 67, 1979). PKB in turn might act as a functional mediator of Twist-1 and is involved in Twist-mediated chemotherapeutic drug resistance (Cheng et al., 2007, Cancer Res, 67, 1979; Zhang et al., 2007, IntJCancer, 120, 1891). Interestingly, SCS resulting from Twist-1 haploinsufficiency displays decreased expression of Cbl ubiquitin ligase, resulting in the accumulation of PI3K and increased PI3K/PKB signaling (Guenou, Kaabeche et al., 2006, AmJPathol, 169, 1303).

https://connect.fmi.ch/,DanaInfo=www.sciencedirect.com+science? ob=ArticleURL& udi=B6 WSN-4W9297H-9& user=5234439& rdoc=1& fmt=& oriq=search& sort=d& docanchor=&view=c& acct=C 000006118& version=1& urlVersion=0& userid=5234439&md5=7fa9917c68098913a411c6 d3e9a336dd-aff1

The present inventors therefore investigated whether there is an interaction between PKB and Twist1. In the present disclosure, the present inventors show that PKB kinase becomes activated and phosphorylates transcription factor Twist-1 at serine 42 in MCF-7 cells following serum stimulation, γ-irradiation or DNA damage induced by adriamycin. The present inventors noted that this posttranslational modification of Twist-1 is necessary for the subsequent decrease in total p53 level and the inhibition of cell cycle arrest and apoptosis via impaired activation of p53 target genes. Moreover, the present inventors found that Twist-1 Ser42 phosphorylation occurs in particular human cancers, especially colorectal, breast, lung and prostate. The results presented in the present disclosure thus provide evidence that Twist-1 is a novel PKB nuclear substrate and establish a link between PKB activation and the downregulation of the p53 tumor suppressor. Moreover, the present inventors also found that Twist1 phosphorylation promotes EMT and metastasis.

The present invention thus encompasses a method for treating cancer in a subject by modulating the phosphorylation of the Serine 42 of Twist1 by administering to said subject a therapeutically effective amount of a modulator of said phosphorylation of the Serine 42 of Twist1. In some embodiments, the phosphorylation of the Serine 42 of Twist1 is modulated by an inhibitor which specifically binds to Twist1 and hinders, e.g. by allosteric hindrance, the phosphorylation of its Serine 42 by PKB., for instance an antibody or a small molecule. In some embodiments of the invention, the subject is a mammal, for instance a human subject. In some embodiments, the method of the invention is performed in vivo, ex vivo or in vitro.

In some embodiments of the invention, the epithelial-mesenchymal transition (EMT) of cancer cells and/or metastasis formation is reduced. In some embodiments, the cancer is a melanoma, a colorectal cancer, a breast cancer, a lung cancer or a prostate cancer.

The present invention also encompasses an antibody or a small molecule specifically binding to Twist1 and hindering the phosphorylation of the Serine 42 of Twist1 by PKB, for use as a medicament to treat cancer. In some embodiments, the antibody of the invention specifically binds to an epitope of Twist1, which epitope comprises the Serine 42 of Twist1.

In some embodiments, fragments of the Twist1 protein, which fragments comprise an amino acid corresponding to the Serine 42 of Twist1 and is recognized and phosphorylated by PKB, can be used to modulate the phosphorylation of the Serine 42 of Twist1 by PKB. Example of such fragments are described herein below, for instance those of SEQ ID NO:1, 3 or 5. These fragments, as well as their uses, are also encompassed by the present invention. The size of such a fragment, polypeptide or peptide, will typically be between 5 and 50 amino acids long, for instance between 10 and 30 amino acids long, for example 15, 16, 17, 18, 19, 20, 21, 22, 23, 24, 25, 26, 27, 28 or 29 amino acids long. Typically such a fragment, polypeptide or peptide, will have at least 80%, 85%, 90%, 92%, 95%, 96%, 97%, 98%, 99%, or 100% identity to the corresponding amino acid sequence of Twist1. In addition, the present invention also encompasses nucleic acid molecules encoding for such fragments, vectors comprising said nucleic acid molecules and cells comprising such vectors.

The present invention also encompasses a method for the identification of a substance that modulates a PKB signaling pathway, e.g. the PI3K/PTen/mTor/PKB or the PI3K/PTen/DNAPK/PKB pathway, which method comprises the step of assessing the phosphorylation of the Serine 42 of Twist1.

Another aspect of the present invention is a method of diagnosing cancer comprising the step of assessing the phosphorylation of the Serine 42 of Twist1. In some embodiments, an increased phosphorylation of the Serine 42 of Twist1 is indicative of a potential epithelial-mesenchymal transition of cancer cells and/or metastasis formation. Moreover, an increased phosphorylation of the Serine 42 of Twist1 can also be indicative of a potential resistance to chemotherapeutic drugs.

A further aspect of the present invention encompasses the use of the phosphorylation of the Serine 42 of Twist1 as a biomarker for cancer or to stratify cancer patients. Moreover, the metastatic potential of the cancer can be assessed according to this aspect of the present invention.

Furthermore, the present invention also provides kits comprising means to detect and/or assess the phosphorylation of the Serine 42 of Twist1. Said mean can e.g. be an antibody as described herein-above. These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically. In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml/salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Aced Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5 and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5 and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence. As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=I, Window Size=sequence length, Gap Penalty--5, Gap Size Penalty--0.05, Window Size=500 or the length of the subject amino acid sequence, whichever is shorter. If the subject sequence is shorter than the query sequence due to N-or C-terminal deletions, not because of internal deletions, a manual correction must be made to the results. This is because the FASTDB program does not account for N-and C-terminal truncations of the subject sequence when calculating global percent identity. For subject sequences truncated at the N-and C-termini, relative to the query sequence, the percent identity is corrected by calculating the number of residues of the query sequence that are N-and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N-and C-termini of the subject sequence, which are not matched/aligned with the query sequence, are considered for the purposes of manually adjusting the percent identity score. That is, only query residue positions outside the farthest N-and C-terminal residues of the subject sequence. Only residue positions outside the N-and C-terminal ends of the subject sequence, as displayed in the FASTDB alignment, which are not matched/aligned with the query sequence are manually corrected for. No other manual corrections are to be made for the purposes of the present invention. Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis. Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-la mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

In one embodiment where one is assaying for the ability to bind or compete with full-length Twist-1 polypeptide for binding to anti-phosphorylated serine 42 of Twist-1 antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. Assays described herein and otherwise known in the art may routinely be applied to measure the ability of polypeptides and variants derivatives and analogs thereof, comprising at least 8 amino acids of Twist-1, wherein one of said amino acids corresponds to the serine 42 of Twist-1, to elicit Twist-1-related biological activity (either in vitro or in vivo) and/or to assess whether Twist-1 is present in a given sample, e.g. a sample isolated from a patient.

The term “epitopes,” as used herein, refers to portions of a polypeptide having antigenic or immunogenic activity in an animal, in some embodiments, a mammal,for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CHI, CH2, CH3, or any combination thereof and portions thereof), or albumin (including but not limited to recombinant albumin (see, e.g., U.S. Pat. No. 5,876, 969, issued Mar. 2, 1999, EP Patent 0 413 622, and U.S. Pat. No. 5,766,883, issued June 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e. g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834, 252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Antibodies of the invention include, but are not limited to, polyclonal, monoclonal, multispecific, human, humanized or chimeric antibodies, single chain antibodies, Fab fragments, F(ab') fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgGI, IgG2, IgG3, IgG4, IgA1 and IgA2) or subclass of immunoglobulin molecule.

In addition, in the context of the present invention, the term “antibody” shall also encompass alternative molecules having the same function, e.g. aptamers and/or CDRs grafted onto alternative peptidic or non-peptidic frames. In some embodiments the antibodies are human antigen-binding antibody fragments and include, but are not limited to, Fab, Fab' and F(ab')2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4, 474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811, 097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17)11295-11301 (1997); Taryman et al., Neuron 14(4):755-762 (1995); Muller et al., Structure 6(9):1153-1167 (1998); Bartunek et al., Cytokine 8(1):14-20 (1996).

As discussed in more detail below, the antibodies may be used either alone or in combination with other compositions. The antibodies may further be recombinantly fused to a heterologous polypeptide at the N-or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain. For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5, 698,426; 5,223,409; 5,403,484; 5,580,717; 5,427,908; 5,750,753; 5,821, 047; 5,571, 698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab' and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716, 111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569, 825; 5, 661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies. Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et aL, Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676, 980.

By “affinity” as used herein is meant the propensity of one chemical species to separate or dissociate reversibly from another chemical species. In the present invention, the two chemical species most typically are represented by a protein and its ligand, more specifically an antibody and its target antigen. Affinity herein is measured by the equilibrium constant of dissociation (Kd or Kd) that defines the binding between the two chemical species. The Kd defines how tightly the species bind one another. The smaller the dissociation constant, the more tightly bound the ligand is, or the higher the affinity between ligand and protein. For example, an antigen with a nanomolar (nM) dissociation constant binds more tightly to a particular antibody than a ligand with a micromolar (μM) dissociation constant. By “greater affinity” or “improved affinity” or “enhanced affinity” or “better affinity” than a parent polypeptide, as used herein is meant that a protein variant binds to its ligand with a significantly higher equilibrium constant of association (KA or Kₐ) or lower equilibrium constant of dissociation (Kd or K_{d}) than the parent protein when the amounts of variant and parent polypeptide in the binding assay are essentially the same. For example, in the context of antibodies, a variant antibody may have greater affinity to the antigen that its parent antibody, for example when the CDRs are humanized, as described herein. Alternatively, and Fc polypeptide may have greater affinity to an Fc receptor, for example, when the Fc variant has greater affinity to one or more Fc receptors or the FcRn receptor. In general, the binding affinity is determined, for example, by binding methods well known in the art, including but not limited to BiacoreTM assays. Accordingly, by “reduced affinity” as compared to a parent protein as used herein is meant that a protein variant binds its ligand with significantly lower Ka or higher Kd than the parent protein. Again, in the context of antibodies, this can be either to the target antigen, or to a receptor such as an Fc receptor. Greater or reduced affinity can also be defined relative to an absolute level of affinity. For example, greater or enhanced affinity may mean having a Kd lower than about 10 nM, for example between about 1 nM-about 10 nM, between about 0.1-about 10 nM, or less than about 0.1 nM.

The term “specifically binds” refers, with respect to an antigen to the preferential association of an antibody or other ligand, in whole or part, with a cell or tissue bearing that antigen and not to cells or tissues lacking that antigen. It is recognized that a certain degree of non-specific interaction can occur between a molecule and a non-target cell or tissue. Nevertheless, specific binding can be distinguished as mediated through specific recognition of the antigen. Although selectively reactive antibodies bind antigen, they can do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a specific antigen as compared to an unspecific antigen. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity. The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating cancer in a subject by inhibiting the phosphorylation of the serine 42 of Twist-1 by administration to the subject of an effective amount of an inhibitory compound or pharmaceutical composition comprising such inhibitory compound. In some embodiments, said inhibitory compound is an antibody. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)).

Other controlled release systems are discussed in the review by Langer (Science 249:1527-1533 (1990)). The present invention also provides pharmaceutical compositions for use in the treatment of cancer by inhibiting the phosphorylation of the serine 42 of Twist-1. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection. Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for examplel mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643, 575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,53 1; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

The inhibitors of the phosphorylation of the serine 42 of Twist-1 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site. The inhibitors of the phosphorylation of the serine 42 of Twist-1 may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted at the site of a tumour, and the molecule may be released over weeks or months. Such devices may be particularly advantageous when long term treatment with an inhibitor of the phosphorylation of the serine 42 of Twist-1 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration.

Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 pg/kg of body weight and 0.5 g/kg of body weight of an inhibitor of the phosphorylation of the serine 42 of Twist-1 may be used for the treatment of cancer in the subject, depending upon which specific inhibitor is used. When the inhibitor is delivered to a cell, daily doses may be given as a single administration (e.g. a single daily injection).

Various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term “cancer” refers to a group of diseases in which cells are aggressive (grow and divide without respect to normal limits), invasive (invade and destroy adjacent tissues), and sometimes metastatic (spread to other locations in the body). These three malignant properties of cancers differentiate them from benign tumors, which are self-limited in their growth and don't invade or metastasize (although some benign tumor types are capable of becoming malignant). A particular type of cancer is a cancer forming solid tumours. Such cancer forming solid tumours can be breast cancer, prostate carcinoma or oral squamous carcinoma. Other cancer forming solid tumours for which the methods and inhibitors of the invention would be well suited can be selected from the group consisting of adrenal cortical carcinomas, angiomatoid fibrous histiocytomas (AFH), squamous cell bladder carcinomas, urothelial carcinomas, bone tumours, e.g. adamantinomas, aneurysmal bone cysts, chondroblastomas, chondromas, chondromyxoid fibromas, chondrosarcomas, fibrous dysplasias of the bone, giant cell tumours, osteochondromas or osteosarcomas, breast tumours, e.g. secretory ductal carcinomas, chordomas, clear cell hidradenomas of the skin (CCH), colorectal adenocarcinomas, carcinomas of the gallbladder and extrahepatic bile ducts, combined hepatocellular and cholangiocarcinomas, fibrogenesis imperfecta ossium, pleomorphic salivary gland adenomas head and neck squamous cell carcinomas, chromophobe renal cell carcinomas, clear cell renal cell carcinomas, nephroblastomas (Wilms tumor), papillary renal cell carcinomas, primary renal ASPSCR1-TFE3 t(X;17)(p11;q25) tumors, renal cell carcinomas, laryngeal squamous cell carcinomas, liver adenomas, hepatoblastomas, hepatocellular carcinomas, non-small cell lung carcinomas, small cell lung cancers, malignant melanoma of soft parts, medulloblastomas, meningiomas, neuroblastomas, astrocytic tumours, ependymomas, peripheral nerve sheath tumours, neuroendocrine tumours, e.g. phaeochromocytomas, neurofibromas, oral squamous cell carcinomas, ovarian tumours, e.g. epithelial ovarian tumours, germ cell tumours or sex cord-stromal tumours, pericytomas, pituitary adenomas, posterior uveal melanomas, rhabdoid tumours, skin melanomas, cutaneous benign fibrous histiocytomas, intravenous leiomyomatosis, aggressive angiomyxomas, liposarcomas, myxoid liposarcomas, low grade fibromyxoid sarcomas, soft tissue leiomyosarcomas, biphasic synovial sarcomas, soft tissue chondromas, alveolar soft part sarcomas, clear cell sarcomas, desmoplastic small round cell tumours, elastofibromas, Ewing's tumours, extraskeletal myxoid chondrosarcomas, inflammatory myofibroblastic tumours, lipoblastomas, lipoma, benign lipomatous tumours, liposarcomas, malignant lipomatous tumours, malignant myoepitheliomas, rhabdomyosarcomas, synovial sarcomas, squamous cell cancers, subungual exostosis, germ cell tumours in the testis, spermatocytic seminomas, anaplastic (undifferentiated) carcinomas, oncocytic tumours, papillary carcinomas, carcinomas of the cervix, endometrial carcinomas, leiomyoma as well as vulva and/or vagina tumours. In an embodiment of the invention, the cancer is a colorectal cancer, a breast cancer, a lung cancer or a prostate cancer.

As used herein, the tem “metastasis” refers to the spread of cancer cells from one organ or body part to another area of the body, i.e. to the formation of metastases. This movement of tumor growth, i.e. metastasis or the formation of metastases, occurs as cancer cells break off the original tumor and spread e.g. by way of the blood or lymph system. Without wishing to be bound by theory, metastasis is an active process and involves an active breaking from the original tumor, for instance by protease digestion of membranes and or cellular matrices, transport to another site of the body, for instance in the blood circulation or in the lymphatic system, and active implantation at said other area of the body.

In one embodiment, the cancer is a cancer dependent on the phosphorylation of the serine 42 of Twist-1. Cancers dependent on the phosphorylation of the serine 42 of Twist-1 are cancers where the phosphorylation of the serine 42 of Twist-1 has become essential. Cancers dependent on the phosphorylation of the serine 42 of Twist-1 can be easily identified by inhibiting the phosphorylation of the serine 42 of Twist-1, and identifying the cancers that are not able to grow, migrate or forming metastases in the absence of it.

The present invention also provides a method of screening compounds to identify those which might be useful for treating cancer in a subject by inhibiting the phosphorylation of the serine 42 of Twist-1 as well as the so-identified compounds.

As used herein and as in the fields of pharmacology and biochemistry, a “small molecule” is a low molecular weight organic compound which is by definition not a polymer. The term small molecule is restricted to a molecule that also binds with high affinity to a biopolymer such as protein, nucleic acid, or polysaccharide and in addition alters the activity or function of the biopolymer. The upper molecular weight limit for a small molecule is approximately 800 Daltons which allows for the possibility rapid diffuse across cell membranes so that they can reach intracellular sites of action. In addition, this molecular weight cutoff is necessary but insufficient condition for oral bioavailability. Small molecules can have a variety of biological functions, serving as cell signalling molecules, as tools in molecular biology, as drugs in medicine, and in countless other roles. These compounds can be natural (such as secondary metabolites) or artificial (such as antiviral drugs); they may have a beneficial effect against a disease (such as drugs) or may be detrimental (such as teratogens and carcinogens). Biopolymers such as nucleic acids, proteins, and polysaccharides (such as starch or cellulose) are not small molecules, although their constituent monomers—ribo- or deoxyribonucleotides, amino acids, and monosaccharides, respectively—are considered to be. Very small oligomers are also considered small molecules, such as dinucleotides, peptides such as the antioxidant glutathione, and disaccharides such as sucrose.

“Twist-1”, also refered to as Twist1, twist1, twist-1, ACS3, BPES2, BPES3, H-twist, SCS, TWIST, bHLHa38, twist, B-HLH DNA binding protein, TWIST homolog of drosophila, acrocephalosyndactyly 3, blepharophimosis, epicanthus inversus and ptosis 3, and twist homolog 1 (Drosophila), refers to a transcription factor which is a basic-helix-loop-helix transcription factor associated with Saethre-Chotzen syndrome. Basic helix-loop-helix (bHLH) transcription factors have been implicated in cell lineage determination and differentiation. The protein encoded by this gene is a bHLH transcription factor and shares similarity with another bHLH transcription factor, Dermo1. The strongest expression of this mRNA is in placental tissue; in adults, mesodermally derived tissues express this mRNA preferentially. Mutations in this gene have been found in patients with Saethre-Chotzen syndrome. The amino acid sequence of human Twist-1 is that of SEQ ID NO:9. AKT protein family, which members are also called protein kinases B (PKB) plays an important role in mammalian cellular signaling. In humans, there are three genes in the “Akt family”: Akt1, Akt2, and Akt3. These genes code for enzymes that are members of the serine/threonine-specific protein kinase family (EC 2.7.11.1).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

### Materials and Methods

### Cell Culture, Transfection and Stimulation

Human HEK293, MCF7 and H1299 cells were grown in Dulbecco's modified Eagle medium (DMEM) supplemented with 10% (v/v) fetal calf serum (FCS), 2 mM L-glutamine and 1% (v/v) penicillin/streptomycin. All cells were grown in a humidified incubator at 37° C. and 5% CO2. Cells were plated 24 h prior to transfection and transiently transfected using jetPEI (PolyPlus Transfection) or Lipofectamine 2000 (Invitrogen) according to the manufacturers instructions. DNA amounts were standardized by addition of empty expression vector. HEK293 cells were starved in DMEM containing no serum for 24 h prior to stimulation with 20% FCS for 1 h; LY 294002 inhibitor was added 30 min prior to stimulation as indicated. MCF7 cells were γ-irradiated with the indicated doses 24-36 hours after transfection (TORREX 120D, Astrophysics Research Corp.).

### Generation of Stable MCF7 Cell Lines by Retroviral Infection

To produce retrovirus vectors, BOSC retrovirus packaging cells were transiently transfected with the retroviral pBABEpuro empty vector or pBABEpuro Twist-1 constructs by the calcium phosphate method. Viral supernatants were harvested 48 h after transfection, filtered through a 0.45 μm membrane and applied to MCF7 cells in 10 cm dishes with 5 pg/ml polybrene (Sigma). A second infection was performed 8-12 h later. At 24 h after retroviral infection, cells were selected with 3-5 μg/ml puromycin (Sigma) for 6-8 days and resistant clones were propagated.

### Antisera, Plasmids and Reagents

Flag-hTwist-1, Myc-hTwist-1 (both cloned via BamH1/Xho1 in pcDNA3), GST-hTwist-1 (via EcoR1 in pGex4T.3) were cloned using standard PCR procedures with the full-length cDNA of the IRAUp969H1277D clone (Rzpd, Germany) as a template. Point mutations were introduced by PCR using the QuikChange site mutagenesis protocol (Stratagene). shRNA constructs were cloned into the pTER vector. Targeting sequences used for generating shRNA against human PKB and firefly luciferase were as described previously (Bozulic et al., 2008, Mol Cell, 30, 203; Vichalkovski, Gresko et al., 2008, Curr Biol, 18, 1889). The reporter plasmids p21^{waf1}-Luc (el-Deiry, Tokino et al., 1993, Cell, 75, 817) and Bax-Luc (Fogel, Gostissa et al., 2000, EMBO J, 19, 6185) were as published, E-cadherin-Luc construct was a kind gift of A. DiFeo (The Mount Sinai School of Medicine). Antibodies recognizing total PKB, phospho-PKB (Ser473), p21^{Waf1} and the phospho-(Ser/Thr) PKB substrate antibody were purchased from Cell Signaling Technologies; anti-p53 (DO-1) and anti-actin antibodies were from Santa Cruz Biotechnology. A rat monoclonal anti-tubulin (YL1/2) and mouse anti-Myc-9E10 were used as hybridoma supernatants; the antibody against Flag (M2) was from Sigma. Anti-Mdm2 antibody was described previously (Feng et al., 2004, JBiolChem, 279, 35510). Anti-Twist-P-Ser42 and anti-Twist-P-Ser123 rabbit polyclonal antibodies were raised against synthetic peptides conjugated to keyhole limpet hemocyanin: CGGRKRRSS(PO3H2)RRSAGG (SEQ ID NO:1) peptidefor the Ser-42 phosphorylation site and CNVRERQRTQS(PO3H2)LNEA (SEQ ID NO:2) peptidefor the Ser-123 phosphorylation site. Peptide synthesis, rabbit injection and bleed collection were carried out by NeoMPS (Strasbourg, France). The antibodies were then purified on the corresponding antigenic peptides coupled to cyanogen bromide-activated Sepharose (Amersham Biosciences). Antibodies were eluted with 0.2 M glycine, pH 2.2. Antibody specificity was confirmed by standard peptide competition. Briefly, an aliquot of the purified antibody was incubated with the phosphopeptide (at 0.5 mg/ml final concentration) in TBS buffer for 2 h on ice with agitation prior to Western blotting. Polyclonal antibody recognizing total Twist-1 was raised in rabbits against full-length GST-fusion Twist-1 (Eurogentec, Belgium). Antisera were affinity-purified using immobilized antigen and extensively characterized.

### Immunoprecipitation and Western Blotting

For Western blot analysis, cells were lysed in lysis buffer containing 50 mM HEPES pH 7.5, 1% (v/v) Triton X-100, 50 mM NaCl, 5 mM EGTA, 50 mM sodium fluoride, 20 mM sodium pyrophosphate, 1 mM sodium vanadate, 2 mM PMSF and 0.2 μg/ml aprotinin and leupeptin. Lysates were cleared by centrifugation at 13,000 rpm for 10 min at 4° C. Supernatants (20-50 pg per sample) were resolved by SDS gel electrophoresis and then transferred to Immobilon-P polyvinylidene difluoride membranes (Millipore), incubated with the corresponding antibodies and developed with either horseradish peroxidase-coupled (Amersham Biosciences) or infrared-labeled secondary antibodies (Rockland Immunochemicals) and quantified by the Odyssey imaging system. For immunoprecipitation, 300 μg of total protein lysate was preincubated with 1.3 μg of anti-Flag antibodies for 3 h at 4° C. with rotation, followed by addition of protein A/G agarose (GE Healthcare) for a further 6 h.

In vitro Kinase Assays on Peptides and GST-Fusion Proteins

The peptides for in vitro kinase assay were synthesized by NeoMPS and further purified (Franz Fischer, FMI). For a kinase reaction, 2 μl (100 ng) of the activated or inactivated recombinant PKBβ (Yang, Cron et al., 2002, MolCell, 9, 1227) was added to a reaction mix containing 70 μM of the corresponding peptide (RKRRSSRRSAGG—S42/S45 (SEQ ID NO:3), RKRRSARRSAGG—S42A (SEQ ID NO:4), RKRRSSRRAAGG—S45A (SEQ ID NO:5), RERQRTQSLNEA—T121/5123 (SEQ ID NO:6), RERQRAQSLNEA—T121A (SEQ ID NO:7), RERQRTQALNEA—S123A (SEQ ID NO:8)), 2 μl (2 μCi) of γ-³²P-ATP and 20 μM ATP in 20 μl of kinase reaction buffer (30 mM HEPES/KOH pH 7.4, 25 mM β-glycerophosphate, 2 mM DTT, 20 mM MgCl₂, 0.1 mM sodium vanadate). After incubation for 30 min at 30° C., kinase reactions were stopped with 50 mM EDTA, transferred to phosphocellulose P11 paper (Whatman), fixed and washed four times in 1% phosphoric acid and once with acetone, dried and assayed by scintillation counting.

GST-Twist-1 or its point mutants (S42A, S123A and SS42, 123/AA) were purified from bacterial strain BL-21 according to a standard protocol. For in vitro kinase assays, 5-10 μg of GST fusion protein was incubated with 100 ng of the recombinant PKBβ in the presence of 20 μM ATP in 25 μl of kinase reaction buffer for 30 min at 30° C. The reaction was stopped by adding SDS sample buffer and protein phosphorylation was analyzed by SDS-PAGE and Western blotting with the phospho-(Ser/Thr) PKB substrate, anti-Twist-P-Ser42 and anti-Twist-P-Ser123 antibodies or by capillary liquid chromatography tandem mass spectrometry (LC-MSMS, see below).

### LC-MSMS Analysis of GST-Twist-1 Phosphorylation

The protein spots were excised from the gel, reduced with 10 mM DDT, alkylated with 55 mM iodoacetamide and cleaved with porcine trypsin (Promega, Madison, USA) or lysyl endopeptidase (Wako, Osaka, Japan) in 50 mM ammonium bicarbonate (pH 8.0) at 37° C. overnight (Shevchenko, Wilm et al., 1996, Anal Chem, 68, 850). The extracted peptides were analyzed by LC-MSMS using a Magic C18 100 μm×10 cm HPLC column (Spectronex, Switzerland) connected online to a 4000 Q Trap (MDS Sciex, Concord, Ontario, Canada). A linear gradient from 5% to 45% B (0.1% formic Acid, 80% acetonitrile in H₂O) in A (0.1% formic acid, 2% acetonitrile in H₂O) was delivered with an 1100 Nano-HPLC system (Agilent, Palo Alto, Calif.) at 300 nl/min in 45 min. The peptides were loaded for 5 min at a flow of 10 μl/min in 5% buffer B onto a peptide captrap (Michrom BioResources, Inc. Calif.). The eluting peptides were subjected to electrospray ionization. The masses of the peptide ions were measured in the linear ion trap, then the detected ions were automatically selected in Quadrupol 1, fragmented in Quadrupol 2 and the generated ions were measured in the linear ion trap (Hess, Keusch et al., 2008, J Biol Chem, 283, 7354). For phosphopeptide identification, individual MSMS spectra containing sequence information for a single peptide were compared using Mascot (Perkins, Pappin et al., 1999, Electrophoresis, 20, 3551) to the protein sequence database UNIPROT_{—}8.6. of May 2007 (3413450 sequences; 1115317345 residues). Uniprot is the combined SwissProt and TrEMBL protein sequence database. For improved sequence coverage, dedicated MSMS spectra were acquired for individual phosphopeptides.

### Immunohistochemistry

Paraffin-embedded slides of whole mouse embryos (E14.5) and sections of paraffin-embedded tissue microarray slides (MC2081, MCN601) (US Biomax Inc, Rockville, USA) were stained with anti-Twist-P-Ser42 antibody and counterstained with hematoxylin-eosin using standard protocol for the Discovery XT Staining Module (Ventana). Images were processed with a Nikon E600 microscope system.

### Confocal Microscopy

MCF7 cells seeded on coverslips were fixed for 1 min at −20° C. with methanol/acetone (1:1), air-dried, rehydrated with PBS, blocked with goat serum and incubated with appropriate antibodies. Image stacks were recorded with an Olympus FV500 laser scanning microscope and Fluoview 1000V.1 application software.

### Luciferase Reporter Gene Assays

Harvested cells were lysed in reporter lysis buffer (25 mM Tris-Phosphate, 2 mM DTT, 2 mM CDTA, 10% (v/v) glycerol, 1% (v/v) Triton X-100). Luciferase activity was determined in a luminometer (Duo Lumat LB 9507, Berthold) by injecting 20 μl of assay buffer (40 mM Tricine, 2.14 mM (MgCO₃)4 Mg(OH)2×5 H₂O, 5.34 mM MgSO₄, 0.2 mM EDTA, 66.6 mM DTT, 540 M CoA, 940 μM luciferin, 1.06 mM ATP) and measuring light emission for 10 s.

### Cell Cycle Analysis and Apoptosis Measurement

For FACS analysis of DNA content, cells were trypsinized, fixed in 70% ice-cold ethanol, then treated with RNase A (10 μg) in propidium iodide (PI) solution (sodium citrate [pH 7.5], 69 μM PI) for 30 min at 37° C. and analyzed using a FACSCalibur flow cytometer (Becton Dickinson). Cells undergoing apoptosis were harvested, washed with PBS and subdivided into two fractions. One fraction was stained with JC-1 (5,5′,6,6′-tetrachloro-1,1′,3,3′-tetraethylbenzimidazolylcarbocyanine iodide) according to the manufacturer's instructions (Molecular Probes) and subjected to flow cytometry for detection of mitochondrial depolarization (ΔψPm). Red fluorescence (FL-2 channel) of JC-1 (J-aggregates) indicated intact mitochondria, whereas green fluorescence (FL-1 channel) showed monomeric JC-1 produced by break-down of Δψm during apoptosis. The remaining cells were analyzed by western blotting.

### Results

PKB Phosphorylates Twist-1 in vitro at Ser42 and Ser123

PKB signaling pathway is one of the most frequently altered in human cancer (Franke, 2008, Oncogene, 27, 6473; Yoeli-Lerner and Toker, 2006, Cell Cycle, 5, 603), and yet there are only few data directly implicating downstream targets of PKB in an oncogenic switch and cancer progression. As transcription factor Twist-1 was proposed recently to be a potent inducer of malignant transformation, and despite the fact that it had been reported that Twist1 is phosphorylated by PKA (Firulli, Krawchuk et al., 2005, NatGenet, 37, 373), the present inventors examined whether this protein is a PKB substrate. Four sites in human Twist-1 (Ser 42, Ser45, Thr121 and Ser123) have been predicted to be phosphorylated by PKB. Three of them (S42, T121 and S123) display the canonical PKB substrate consensus motive: K/RXK/RXXS/T. Two N-terminal sites are situated in the low-complexity region of the molecule, whereas Thr121 and Ser123 lie within the bHLH domain, responsible for dimerization and DNA binding activity of Twist-1. The present inventors tested the ability of recombinant PKBβ to induce phosphorylation of synthetic peptides comprising PKB recognition motifs and corresponding phosphosites, as well as their mutated analogs Ser42A, Ser45A, Thr121A and Ser125A. Only substitution of serine to alanine at positions 42 and 123 resulted in almost complete loss of phosphorylation of the corresponding peptide by PKB. Next, the present inventors tested the ability of PKB to phosphorylate the full-length Twist-1 protein. To control the specificity of the in vitro kinase reaction an inactive PKBβ was used. To further investigate which of the potential sites were preferentially phosphorylated in the full-length Twist-1, the present inventors performed a series of in vitro kinase assays followed by mass spectrometry (MS) analysis. This identified two phosphopeptides corresponding to Twist-1 amino acid sequences containing S42 and S123 phosphosites. A more detailed MS analysis using inactive PKBβ and recombinant Twist-1 mutants confirmed that PKB phosphorylates Twist-1 in vitro on two residues S42 and S123.

PKB Phosphorylates Twist-1 at Ser42 in vivo

As their results indicated that PKB phosphorylates Twist-1 in vitro, the present inventors examined whether this is also the case in cell culture. Immunoprecipitated Twist-1 was detected with the pan-PKB phosphosubstrate antibody in serum-stimulated but not in starved HEK293 cells. Moreover, pre-treatment of the cells with an inhibitor of the PI3K-PKB pathway (LY 294002) resulted in a strong reduction in the phospho-signal, suggesting a specific phosphorylation by PKB. Treatment of cells with lambda phosphatase almost completely abolished Twist-1 phosphorylation, confirming that Twist-1 exists as a phosphoprotein in cells. Importantly, since phosphorylation of the Twist-1 S42A mutant was not detected but the S123A mutant was phosphorylated as efficiently as the wild-type Twist-1, the results suggested preferential phosphorylation of S42 in vivo. Nevertheless, the pan-PKB phosphosubstrate antibody recognized Twist-1 phosphorylated on S42 or Ser123 in vitro equally well, indicating that the antibody is capable of detecting both phosphosites.

To study the function of PKB-mediated Twist-1 phosphorylation in cells, the present inventors generated antibodies against the two phosphosites S42 and S123. Thorough characterization confirmed the phosphospecificity of the antibodies in the in vitro kinase assay using wild-type or mutant Twist-1 proteins as substrates. To verify that Twist-1 can be phosphorylated under physiological conditions, starved HEK293 cells were stimulated with serum to induce PKB activity. Importantly, in these conditions phosphospecific antibodies detected Twist-1 only when phosphorylated at S42 but not at S123. As mentioned, it had been shown that S123 can be phosphorylated by PKA (Firulli and Conway, 2008, Curr Med Chem, 15, 2641). Indeed, stimulation of cells with forskolin resulted in phosphorylation of Twist-1 at S123, which was also detected by our anti-Twist-P-Ser123 antibody, thus confirming its specificity. Altogether, these data indicate that PKB preferentially phosphorylates Twist-1 at S42 in cells. To further illustrate specific role for PKB in the regulation of Twist-1 phosphorylation, the level of endogenous PKB kinase was decreased using shRNA. Twist-1 S42 phosphorylation was not induced after serum stimulation of cells with a low PKB content.

PKB Phosphorylation of Twist-1 at Ser42 Regulates Twist-1-mediated Inhibition of the p53 Response Upon DNA Damage

Taking into account the role of PKB as a pro-survival factor and the recent finding of some of the inventors that PKB can be activated in the nucleus in response to DNA double-strand breaks (Bozulic et al., 2008, Mol Cell, 30, 203), the present inventors hypothesized that phosphorylation of Twist-1 at S42 plays a role in promoting cell survival after DNA damage-induced stress. To test this hypothesis the present inventors knocked-down endogenous PKB in MCF7 cells (human breast cancer cell line, with a functional p53) expressing Twist-1 and then treated them with γ-irradiation. This resulted in PKB-dependent phosphorylation of Twist-1 at S42. Notably, the expression of wild-type Twist-1 but not the S42A mutant led to a considerable decline in p53 induction upon DNA damage. The effect of the expression of the S42E mutant did not differ from the wild type. Transient expression of Twist-1 and its mutants in MCF7 cells had a similar effect. Following the suppressed p53 response, p21^{Waf1} induction at both transcriptional and protein levels was decreased in the presence of wild type Twist-1 but not of S42A Twist-1 mutant, suggesting a potential role of S42 phosphorylation in cell cycle regulation. Indeed, both wild-type and S42E Twist-1-expressing cells escaped G1 cell cycle arrest, while control cells and cells expressing phospho-deficient S42A Twist-1 accumulated in G1 in response to DNA damage. Quantitative cell cycle analysis showed a significant rescue effect of Twist-1 S42 phosphorylation on G1 phase arrest after γ-irradiation, indicating that S42 phosphorylation confers the ability to progress through the cell cycle even under genotoxic stress.

PKB-Dependent Phosphorylation of Twist-1 at Ser42 is Essential for Twist-1-Mediated Survival after DNA Damage-Induced Stress

Given that activation of PKB and Twist-1 phosphorylation occurred in response to DNA damage and led to impaired induction of p53, the present inventors were prompted to investigate the functional relevance of this phosphorylation in the apoptotic process. For this, they used adriamycin to induce DNA double-strand breaks in MCF7 cells. Cells expressing wild-type or S42E Twist-1 were less prone to develop morphological signs of apoptosis such as membrane blebbing and cellular shrinkage than control cells or cells expressing the S42A Twist-1 mutant. Similarly, in the same experimental conditions, wild-type or S42E Twist-1 expression significantly reduced cleavage of PARP and protection of cells from apoptosis was further confirmed by assessing the mitochondrial membrane potential (ΔΨm). This protection from apoptosis was not observed in the S42A mutant expressing cells. As expected, S42A Twist-1 was also less potent in downregulating the pro-apoptotic p53 transcriptional target Bax. Taken together, these data confirm that phosphorylation of S42 is an important part of Twist-1 mediated anti-apoptotic effects.

Cancer in Various Organs is Associated with Twist-1 Ser42 Phosphorylation

As can be seen from the results presented herein-above, Twist-1 phosphorylation at Ser42 plays a significant role in the overall pro-survival effect of Twist-1. It is also well established that an abnormal cell cycle and resistance to apoptosis are typical hallmarks of cancer. This, together with the inventors' present finding that S42 phosphorylation of Twist-1 promotes cell survival upon genotoxic stress, prompted the present inventors to examine S42 phosphorylation in various tumors.

As it has been shown that Twist-1 is transcriptionally active in developing mouse embryos, the present inventors tested our anti-Twist-P-Ser42 antibody on paraffin embedded sections of mouse embryos. Strong expression was observed in areas known to have active Twist-1 (Gitelman, 1997, DevBiol, 189, 205). The present inventors then applied their anti-Twist-P-Ser42 antibody to stain for phosphorylated Twist-1 on an array of paraffin embedded primary cancer specimens. Remarkably, prominent S42 phosphorylation of Twist-1 was clearly detectable in 50% of 30 colon and 71% of 20 rectal cancers, but not in normal human colorectal tissue. Furthermore 70% of 39 human breast cancer samples tested positive, while a smaller but still significant number of samples were positive in prostate (24%) and lung (35%) cancers.

Collectively, the present inventors' data identify Twist-1 as a novel PKB substrate that becomes phosphorylated by PKB on Ser42 in the N-terminal part of the protein upon serum stimulation and genotoxic stress. This phosphorylation appears to play a significant role in the ability of Twist-1 to downregulate the DNA damage-induced p53 response, thus promoting cell survival, which in turn may result in uncontrolled cell overgrowth and cancer.

The PKB substrate consensus sequence surrounding Ser42 in Twist-1 is evolutionary conserved in vertebrate genomes. In contrast, the Ser 42 residue is not conserved in Hand proteins, the closest relatives of Twist-1 in the HLH family, suggesting that this site is phosphorylated in various species exclusively in Twist-1 and not other HLH transcription factors. Moreover, this phosphorylation may have a function distinct from those of other known Twist-1 phosphosites. Protein kinase A (PKA) phosphorylates two conserved residues within the HLH domain of both Twist1 and Hand2 (T125/5127 and T112/S114, respectively, in mice and T121/5123 and T112/S114—in human) bringing about their dimerization, which is necessary for the regulation of target genes during limb development. A group of Twist-1 mutations identified in patients with SCS was reported to disrupt PKA-mediated phosphorylation, emphasizing the importance of Twist-1 in development (Firulli, Krawchuk et al., 2005, NatGenet, 37, 373). In contrast to the S42A mutation, most mutations within the bHLH domain of Twist-1 negatively affect its transcriptional repressor function (Sosic, Richardson et al., 2003, Cell, 112, 169). Despite Ser42 being located adjacent to a putative NLS of Twist-1 (mutation of Arg39 to Gly present in a patient with mild SCS, results in nuclear exclusion of Twist-1 (Funato, Twigg et al., 2005, HumMutat, 25, 550; Singh and Gramolini, 2009, BMC Cell Biol, 10, 47), the present inventors found that the phosphorylation of Twist-1 by PKB did not influence protein localization.

Even though the relevance of Twist-1 in cancer development has been studied intensively, there are few reports describing its molecular regulation. The present inventors report here that Twist-1 is phosphorylated at Ser42 by PKB (1) in response to serum stimulation of HEK293 cells, (2) in MCF-7 breast cancer cells after 7-irradiation and adriamycin treatment and (3) in human cancer tissues of different origins thereby suggesting that Ser42 phosphorylation is involved in the regulation of cell growth and cell survival upon DNA damage.

Focusing on the molecular events triggered by phosphorylation of Twist-1 by PKB in response to DNA damage, one of the present inventors' key observations is that Ser42 phosphorylation is involved in the downregulation of the p53 tumor suppressor. p53 plays a pivotal role in directing cell responses to various stress stimuli, and p53-controlled transactivation of target genes is an essential feature of each stress-response pathway, although some effects of p53 may be independent of transcription (Kruse and Gu, 2009, Cell, 137, 609). In the present inventors'experiments, decrease in p53 stabilization after DNA damage was paralleled by impaired induction of p21^{Waf1}, but only in cells with up-regulated wild-type Twist-1 and not the S42A Twist-1 mutant. The significant reduction in GO/G1 arrest observed in cells expressing wild-type Twist-1 or the S42E Twist-1 mutant but not in S42A Twist-1 cells provides a functional read out of the inhibitory effect of Twist-1 phosphorylation on the key cell cycle effector p21^{Waf1}. Indeed, Twist1 was shown to override premature senescence via inhibition of p16^{INK4A and} p21^{Waf1} promoter activation induced by H-Ras^{V12} and p53 in E1A-immortalized MEFs: however, the molecular mechanisms involved in this effect are still under investigation (Ansieau, Bastid et al., 2008, Cancer Cell, 14, 79). Further, the present inventors' experiments revealed that the ability of cells to survive significant DNA damage is dependent on Ser42 phosphorylation of Twist-1 and decreased markedly in S42A but not S42E Twist-1-expressing cells. Thus, it appears that phosphorylation of the Twist-1 transcription factor by PKB in response to DNA damage contributes to an anti-apoptotic mechanism. This is in line with the strong pro-survival signaling mediated by PKB kinase. PKB itself is known to increase p53 degradation by physically associating with MDM2 and phosphorylating it at Ser166 and Ser186. This enhances its stability (Feng, Tamaskovic et al., 2004, JBiolChem, 279, 35510), as well as its nuclear localization and interaction with p300, and inhibits its association with pi 9ARF (Zhou et al., 2001, NatCell Biol, 3, 245). Interestingly, the present inventors observed that the expression of the wild type Twist-1 but not of S42A Twist-1 mutant promoted an increase in MDM2 protein levels. Therefore, it remains to be addressed whether the effect of Twist-1 Ser42 phosphorylation on p53 and the induction of its target genes are direct or mediated through other molecules. It was described previously that Twist-1 can inhibit a potent p53 transactivator homeobox protein HOXA5, compromising the p53 response to γ-irradiation via suppressed induction of p21^{Waf1} and inhibition of Ser20 phosphorylation (Stasinopoulos, Mironchik et al., 2005, JBiolChem, 280, 2294). Expression of Twist-1 decreases the level of the p53 upstream activator p14^{ARF}, presumably by affecting production of its mRNA (Kwok, Ling et al., 2007, Carcinogenesis, 28, 2467). Twist-1 binds to and inactivates histone acetyltransferase CBP/p300, which is required to relieve the suppressive effects of chromatin on p53 target genes (Hamamori, Sartorelli et al., 1999, Cell, 96, 405). Without wishing to be bound by theory, the present inventors hypothesize that Twist-1 potentially acts via several independent mechanisms that focus on inhibition of the p53 tumor suppressor pathway. Their hypothesis that Twist-1 Ser42 phosphorylation might be a part of oncogenic signalling during cancer development is further supported by compelling data illustrating the presence of this posttranslational modification in human tumor tissues. The continuing identification of PKB substrates adds to the diverse cellular roles of the kinase, including cell growth, proliferation and survival. As phosphorylation of Twist-1 at Ser42 enhances the ability of transformed cells to circumvent cell cycle arrest or apoptosis, induced by genotoxic stimuli, it might represent one of the mechanisms utilized by cancer cells for uncontrolled growth and survival.

Further data from the inventors indicated that transient expression of both wild-type Twist1 and the phospho-inactive mutant S42A can strongly downregulate E-cadherin expression. However, only wild-type Twist1 promoted the upregulation of EMT-promoting molecules in association with Twist1 phosphorylation. These results showed the potential importance of S42 phosphorylation in driving a full EMT phenotype. Further assays, migration/invasion assays, demonstrated that Twist1-expressing cells exhibit markedly increased motility and invasiveness compared with the S42A mutant upon serum stimulation. PI3K/PKB inhibitors (LY294002 and Wortmannin) not only abolished Twist1 phosphorylation but also strongly retard migration, implying a dependence on PKB activity. Given that Twist1 is active in promoting EMT and metastasis, it was particularly interesting to determine whether Twist1-driven EMT and metastasis requires Twist1 phosphorylation at S42 or not. The present inventors designed a rescue experiment to address this issue. As a first step, they knocked down endogenous Twist1 in 4T1 cells by shRNA and generated the stable cell line 4T1-Tw1KD. Non-specific shRNA targeting luciferase was used as a negative control. Stable clones were selected and analysed for Twist and E-cadherin expression. To explore whether this model cell line can induce lung metastasis, it was injected into Balb/c mice, a standard mouse model to study tumorigenesis and metastasis that rarely develops breast tumour or lung metastasis(Curr Top Microbiol Immunol, 1985, 122, 1). Lung metastases were examined at 2ldays post-injection. In agreement with published data(Yang, Mani, Donaher, Ramaswamy, Itzykson, Come, Savagner, Gitelman, Richardson and Weinberg, Cell, 2004, 117, 927), knockdown of Twist1 in 4T1 cells significantly inhibited 85% of lung metastases compared with the control.

1. A method for treating cancer in a subject by modulating the phosphorylation of the Serine 42 of Twist1 by administering to said subject a therapeutically effective amount of a modulator of said phosphorylation of the Serine 42 of Twist1.

2. The method of claim 1 wherein the phosphorylation of the Serine 42 of Twist1 is modulated by an inhibitor which specifically binds to Twist1 and hinders the phosphorylation of its Serine 42 by PKB.

3. The method of claim 1 wherein the inhibitor is an antibody or a small molecule.

4. The method of claim 1 wherein the modulator is a peptide comprising an amino acid corresponding to the Serine 42 of Twist1, which fragment is recognized and phosphorylated by PKB at said amino acid corresponding to the Serine 42 of Twist1.

5. The method of claim 1 wherein the subject is a mammal.

6. The method of claim 1 wherein the epithelial-mesenchymal transition of cancer cells and/or metastasis formation is reduced.

7. The method of claim 1 wherein the cancer is a colorectal cancer, a breast cancer, a lung cancer or a prostate cancer.

8. (canceled)

9. A peptide comprising an amino acid corresponding to the Serine 42 of Twist1, which peptide is recognized and phosphorylated by PKB at said amino acid corresponding to the Serine 42 of Twist1.

10. A method for the identification of a substance that modulates a PKB signaling pathway, which method comprises the step of assessing the phosphorylation of the Serine 42 of Twist1.

11. A method of diagnosing cancer comprising the step of assessing the phosphorylation of the Serine 42 of Twist1.

12. The method of claim 11, wherein an increased phosphorylation of the Serine 42 of Twist1 is indicative of a potential epithelial-mesenchymal transition of cancer cells and/or metastasis formation.

13. The method of claim 11, wherein an increased phosphorylation of the Serine 42 of Twist1 is indicative of a potential resistance to chemotherapeutic drugs.

14. (canceled)

15. (canceled)

16. The method of claim 5, wherein the mammal is a human.
